(19) United States
(12) Patent Application Publication
(10) Pub. No.: US 2014/0194736 A1
(43) Pub. Date: Jul. 10, 2014

(54) METHOD, APPARATUS AND COMPUTER ACCESSIBLE MEDIUM FOR PROVIDING SIGNAL AND CONTRAST ENHANCEMENT(S) IN OPTICAL IMAGING METHODS

(71) Applicants: Guillermo J. Tearney, Cambridge, MA (US); Tao Wu, Somerville, MA (US); Dongkyun Kang, Somerville, MA (US); Melissa J. Suter, Charlestown, MA (US)

(72) Inventors: Guillermo J. Tearney, Cambridge, MA (US); Tao Wu, Somerville, MA (US); Dongkyun Kang, Somerville, MA (US); Melissa J. Suter, Charlestown, MA (US)

(73) Assignee: THE GENERAL HOSPITAL CORPORATION, Boston, MA (US)

(21) Appl. No.: 14/147,403

(22) Filed: Jan. 3, 2014

Related U.S. Application Data
(60) Provisional application No. 61/749,045, filed on Jan. 4, 2013.
(60) Provisional application No. 61/799,206, filed on Mar. 15, 2013.

(51) Int. Cl. A61B 5/00

(52) U.S. Cl. CPC A61B 5/0059; A61B 5/0073; A61B 5/6861
USPC 600/425; 600/476; 600/431

(57) ABSTRACT

A system for facilitating imaging of an anatomical structure(s) can be provided, and can include, for example, a substance which can induce a change in the anatomical structure(s) when applied to the anatomical structure(s), and an imaging arrangement which can be configured to generate an image of the anatomical structure(s) based on the change of the anatomical structure(s) when the substance is applied. The imaging arrangement can include an apparatus which can be configured to (i) illuminate the anatomical structure(s) with a first electro-magnetic radiation(s), and (ii) receive a second electro-magnetic radiation from the anatomical structure(s) that can be based on the first electro-magnetic radiation. The change can affect a characteristic(s) of the second electro-magnetic radiation.

### CROSS-REFERENCE TO RELATED APPLICATION(S)

This application relates to U.S. patent application Ser. No. 61/749,045 filed Jan. 4, 2013, and U.S. patent application Ser. No. 61/799,206 filed Mar. 15, 2013, the entire disclosures of which are incorporated herein by reference.

### FIELD OF THE DISCLOSURE

The present disclosure relates generally to exemplary embodiments of methods, apparatus and computer-accessible medium for providing enhancing optical signal and image contrast during, e.g., endomicroscopic imaging of normal and diseased tissues, inclduing gastrointestinal tract, respiratory tract, and other internal organs. The exemplary methods, apparatus computer-accessible medium can be utilized and/or performed in conjunction with many optical imaging modelities that use the reflected, scattered, fluorescent, second or higher order harmonic, and sum/difference frequency light from the tissue, e.g., including OCT, reflectance confocal microscopy, Raman micrscopy, and fluorescence microscopy.

### BACKGROUND INFORMATION

The diagnosis of most diseases from gastrointestinal tract, respiratory tract, and other internal organs can be conducted by video endoscopy. During the video endoscopy, regions can be identified for further examination. Biopsies can be taken from these regions and prepared as histological slides. During the histological examination, the morphological changes of the tissue can be examined at a microscopic resolution, and possibly used for the final diagnosis. The diagnosis approach of examining biopsies, however, can be prone to include sampling errors since only a fraction of the region that can have malignant changes can be examined.

An endomicroscopic optical imaging probe has become one of the devices to assess and diagnose diseases arising from the gastrointestinal tract, respiratory tract, and other internal organs. High-resolution microscopic imaging modalities, including optical coherence tomography (OCT), confocal endomicroscopy, fluorescence endomicroscopy, and Raman endomicroscopy, have been shown to visualize key histomorphologic features. Another version of such optical endomicroscopy has been described to facilitate the imaging of the entire organ at a microscopic resolution. For example, optical frequency domain imaging (OFDI) has been described as a high-speed OCT technology that has been used for comprehensive imaging of human distal esophagus. Previous in vivo human imaging studies indicated that OFDI techniques and/or devices can visualize architectural changes of the esophageal tissues that are associated with Barrett's esophagus, low-grade dysplasia, and high-grade dysplasia. Spectrally encoded confocal microscopy (SECM) is a high-speed confocal microscopy technology, and an endoscopic SECM catheter has been developed and used to, e.g., image a large segment of swine esophagus at microscopic resolution in vivo. Furthermore, flexible transbronchial optical frequency domain imaging (TB-OFDI) catheter has been developed to function as a “smart needle” which guides biopsy acquisition to increase the diagnostic yield.

Further, the OFDI imaging can be conducted in an endoscopic capsule format. For example, an OFDI capsule can be swallowed by the un-sedated patient. The OFDI capsule can also provide key morphologic features of the esophageal tissues, normal and diseased. SECM is also being developed as a capsule. Further, a SECM capsule has been provided for swine imaging. The capsule-based endomicroscopic imaging can be conducted as a stand-alone procedure with the need for the video endoscopy and patient sedation. Thus, the capsule-based imaging can reduce the procedural cost and facilitate the use of the endomicroscopic imaging technologies for diagnosis.

Conventional confocal endomicroscopy commonly can use intravenous fluorescent agents to provide image contrast. The use of these exogenous agents can increase the cost and complexity of the imaging procedure. Furthermore, the exogenous fluorescence and the autofluorescence signals inside cells could be lack of contrast due to the relatively homogenously distributed of the signal source, such as the proteins, which are widely dispersed all over the cell. OFDI, reflectance confocal microscopy, Raman microscopy, harmonic and sum/difference frequency generation which detect the scattered light from the tissue as opposed to fluorescence, can be advantageous in that it does not require the administration of fluorescent agents. The natural contrast provided by the light scattering from the cell and tissue, however, can be relatively poor and does not visualize nuclear features of the tissue clearly.

Acetic acid has been used in two photon excitation fluorescence microscopy to increase the contrast of nucleus and cell boundary to the cytoplasm. The enhancements may be due to the interaction of acetic acid with intracellular proteins and gap junction proteins.

Acetic acid further has been used as a cell nucleus contrast agent in third harmonic generation (THG) microscopy. The effect of acetic acid was twofold increased enhancement of cell nuclei relative to lipid bodies, possibly due to acetic acid-induced cross-linking of nucleoprotein with compaction of nuclei and alternation of focal refraction index.

Furthermore, it has been examined the THG and stimulated Raman scattering (SRS) signals within the monolayer cultured living cells during 0.3% acetic acid concentration treatment. The results revealed proteins and/or lipids contributed towards increased optical heterogeneity within cellular cytoplasm and nucleus during acetic acid treatment.

Acetic acid spray has been used in chromoendoscopy to provide improved nuclear contrast for reflectance imaging and enhance the contrast between normal tissues and Barrett's esophagus. Previously, esophageal biopsy samples have been imaged with SECM. The biopsy samples were stained with a diluted acetic acid (concentration=0.6%) before the SECM imaging. The SECM images showed that the use of acetic acid increased the nuclear contrast dramatically, which also improved the visualization of the micro-morphologic changes of the tissue associated with diseases.

To enhance the nuclear contrast in endomicroscopic imaging, the diluted acetic acid can be sprayed during the video endoscopy. Then the endomicroscopic catheters can be introduced to the patient. However, for the capsule-based endomicroscopic imaging, the acetic acid spray is not feasible since the patient does not undergo the video endoscopy. One way of delivering the acetic acid to the patient esophagus is to ask the patient to drink the dilute acetic acid. Acetic acid, however, has pungent smell and may not be easily tolerated by the patient even at a low concentration.

A contrast agent that is based on the daily foods or liquids might have a better chance to be tolerated by the patient.

Accordingly, there may be a need to address at least some of the above-described deficiencies.

### SUMMARY OF EXEMPLARY EMBODIMENTS

It is one of the objects of the present disclosure to provide a safe and tolerable, swallowable formulation for enhancing contrast in scattering and/or fluorescence based endomicroscopic optical imaging of the gastrointestinal tract, respiratory tract, and other internal organs. Such exemplary object can be obtained using, e.g., exemplary embodiments of methods, apparatus and computer-accessible medium for providing enhancing optical scattering and/or fluorescence signal and image contrast during, e.g., endomicroscopic imaging of normal and diseased tissues in gastrointestinal tract, respiratory tract, and other internal organs, according to the present disclosure.

For example, to address the above-described needs, according to one exemplary embodiment of the present disclosure, it is possible to provide apparatus, methods and computer-accessible medium to utilize various foods/liquids as contrast agents that can enhance the nuclear contrast of the tissue for optical imaging. Such exemplary apparatus, methods and computer-accessible medium can be implemented to increase the overall signal level from imaged tissue and/or enhance nuclear contrast for capsule-based endomicroscopic imaging technologies.

According to further exemplary embodiments of the present disclosure, it is possible to use various foods/liquids as oral contrast agents during optical imaging of normal and neoplastic gastrointestinal tissue based on scattering and/or fluorescence signals.

Swine duodenum tissue can be used as a proxy for Barrett's esophagus because it shares the similar structure to Barrett's esophagus. The list of foods/liquids can include one or more of different vinegars, fruit juice, acidic solution, cocktail mix, and sour candies, for example, apple drinking vinegar, honey drinking vinegar, pomegranate drinking vinegar, tamarind drinking vinegar, rice vinegar, Sotaroni vinegar, Delallo Golden Sweet vinegar, Guerzoni Sweet Grape vinegar, Sour Patch Kids™, NeRds®, lemon juice, lime juice, orange juice, tartaric acid, citric acid, malic acid, adipic acid, succinic acid, fumaric acid, hydrochloric acid, cocktail mix, or any other acidic juice or solution which pH value is lower than 5.0.

Various concentrations of Splenda , maple syrup, corn syrup, sugar syrup, sugar, glucose, sucrose, fructose, sucralose, lactose, galactose, maltose, maltodextrin, corn starch, and guar gum are used to thicken the acidic liquids so that the contrast agents can evenly coat and remain on the gastrointestinal tract, respiratory tract, and other internal organs for a longer period time. This can make the contrast agents function more efficiently and provide a larger time window for endomicroscopic imaging.

Various acidic foods/liquids, as well as their combinations of different concentrations of Splenda®, Equal®, Sweet'N Low®, maple syrup corn syrup, sugar syrup, sugar, glucose, sucrose, fructose, sucralose, lactose, galactose, maltose, maltodextrin, corn starch, and guar gum, are applied to swine duodenum tissues.

The contrast agents can be instilled on the duodenal lumen for the liquids or placed on the top of the lumen for the solids. Imaging can be conducted using a SECM benchtop system. Three-dimensional volumes of the tissue can be imaged by SECM and the confocal signals at several depths beneath the tissue surface can be analyzed and compared to the control signal from the tissue without adding any contrast agent at the same imaging depths.

Nuclear signal to background ratio (NSBR) can be quantified by using the following equation: NSBR=(the average intensity in an area of 3×3 μm region of interest from nuclei)/(the average intensity in 3×3 μm region of interest from the nearby tissue).

These and other objects of the present disclosure can be achieved by provision of a system for facilitating imaging of an anatomical structure(s), which can include, for example, a substance which can induce a change in the anatomical structure(s) when applied to the anatomical structure(s), and an imaging arrangement which can be configured to generate an image of the anatomical structure(s) based on the change of the anatomical structure(s) when the substance is applied. The imaging arrangement can include an apparatus which can be configured to (i) illuminate the anatomical structure(s) with a first electro-magnetic radiation(s), and (ii) receive a second electro-magnetic radiation from the anatomical structure(s) that can be based on the first electro-magnetic radiation. The change can affect a characteristic(s) of the second electro-magnetic radiation. The change can be linear or nonlinear optical processes, and can include a reflectance, fluorescence, Raman scattering, and harmonic generation, or a sum/difference frequency generation of the first electro-magnetic radiation(s) to become the second electro-magnetic radiation.

In certain exemplary embodiments of the present disclosure, the imaging arrangement can be a microscopic imaging arrangement, which can operate based on confocal microscopy (SECM), optical coherence tomography (OFDI, SD-OCT, FFOCM), second or higher order harmonic microscopy, sum/difference frequency fluorescence microscopy (one-photon or multi-photon fluorescence), or Raman microscopy (CARS, SRS). The imaging arrangement can have a structure that is configured to be delivered into the anatomical structure(s).

In some exemplary embodiments of the present disclosure, the imaging arrangement has a shape of a pill and can be delivered to the anatomical structure. The length of the pill can be less than 35 mm, and a width of the pill can be less than 15 mm. The imaging arrangement can include a tether that can be connected to the pill, and the imaging arrangement can include a housing which can be coated by the substance. The substance can include acetic acid, tartaric acid, citric acid, malic acid, adipic acid, succinic acid, fumaric acid, hydrochloric acid, cocktail mix, lemon juice, lime juice, orange juice, vinegar, fruit vinegar, and any other acidic juice or solution which pH value is lower than about 5.0. The substance can also be a sour candy.

In some exemplary embodiments of the present disclosure, there can be a further ingestible substance which can have a viscosity which can be higher than that of water, and the substance and the further substance can be mixed prior to ingestion. The further ingestible substance can include maple syrup, corn syrup, sugar syrup, sugar, glucose, sucrose, fructose, sucralose, lactose, galactose, maltose, maltodextrin, corn starch, or guar gum.

According to a further exemplary embodiment of the present disclosure, a further substance can be provided that can be ingested prior to the ingestion of the contrast-altering substance, with the purpose of, e.g., improving the perceived taste of the contrast-altering substance. For example, the fruit of the plant Synsepalum dulcificum, commonly known as “miracle berry” or “miracle fruit”, contains miraculin, a substance which produces a lingering effect when ingested that transforms sour and tart tastes to sweet. Miracle berries or miraculin can be ingested prior to the ingestion of the contrast-altering substance to improve its palatability.

A further object of the present disclosure can include a system that can facilitate imaging of an anatomical structure(s) which can include a first arrangement which can hold a substance therein and can be configured to apply the substance to the anatomical structure(s). Upon application by the first arrangement on the anatomical structure(s), the substance can induce a change in the anatomical structure(s). A second arrangement can be configured to generate an image of the anatomical structure(s) based on the change of the anatomical structure(s) when the substance is applied to the anatomical structure(s) by the first arrangement. The first or second arrangements can be provided in a housing that can have a shape of a pill, and can be configured to be swallowed. In some exemplary embodiments of the present disclosure, the first and second arrangements can be situated in the housing.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 a side cross-sectional view of an acidic liquids flushing device according to a first exemplary embodiment of the present disclosure;

FIG. 2 a side cross-sectional view of an acidic liquids spray device according to a second exemplary embodiment of the present disclosure;

FIG. 3 a side cross-sectional view of an acidic foods/liquids delivery device according to a third exemplary embodiment of the present disclosure;

FIG. 4(a) are exemplary images providing a comparison of an intensity level of SECM signal in swine duodenum tissue after applying Rice Vinegar;

FIG. 4(b) are exemplary images providing a comparison of an intensity level of SECM signal in control tissue without adding any contrast agent;

FIG. 5 is an exemplary SECM image of a swine duodenum tissue after placing Sour Patch Kids™ on top thereof;

FIG. 6 is an exemplary SECM image of the swine duodenum tissue after applying mixed acidic solution of 8 g Splenda® in 4 ml Apple Vinegar;

FIG. 7(a) is an exemplary is an image taken before applying the 100% Lemon Juice to the tissue;

FIG. 7(b) is an exemplary image taken when applying 100% Lemon Juice to the tissue; and

FIGS. 7(c)-7(h) are exemplary images taken at various times (e.g., 7 seconds, 14 seconds, 21 seconds, 28 seconds, 35 seconds, and 42 seconds) after applying the 100% Lemon Juice.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures, or the appended claims.

### DETAILED DESCRIPTION OF EXEMPLARY EMBODIMENTS

For the clinical application, one exemplary method to deliver the acidic foods/liquids can be to let the subject drink or swallow a certain amount of oral contrast agents 30-60 seconds before endomicroscopic imaging of the gastrointestinal tract. The oral agents can be mixed with different concentrations of Splenda®, maple syrup, corn syrup, sugar syrup, sugar, glucose, sucrose, fructose, sucralose, lactose, galactose, maltose, maltodextrin, corn starch, and guar gum to make various viscous acidic solutions, for the purpose of thickening the acidic liquids so that the acidic liquids can coat the gastrointestinal tract, such as the Barrett's esophagus, evenly and last longer to function more efficiently.

In order to apply acidic foods/liquids to the gastrointestinal tract, respiratory tract, and other internal organs, such as the esophagus, according to some exemplary embodiments, it is possible to integrate a miniature flushing/spray duct in the optical imaging catheter such as the OFDI or SECM catheters. The acidic foods/liquids can be delivered to specific locations guided by the optical imaging probes such as the OFDI and SECM probes.

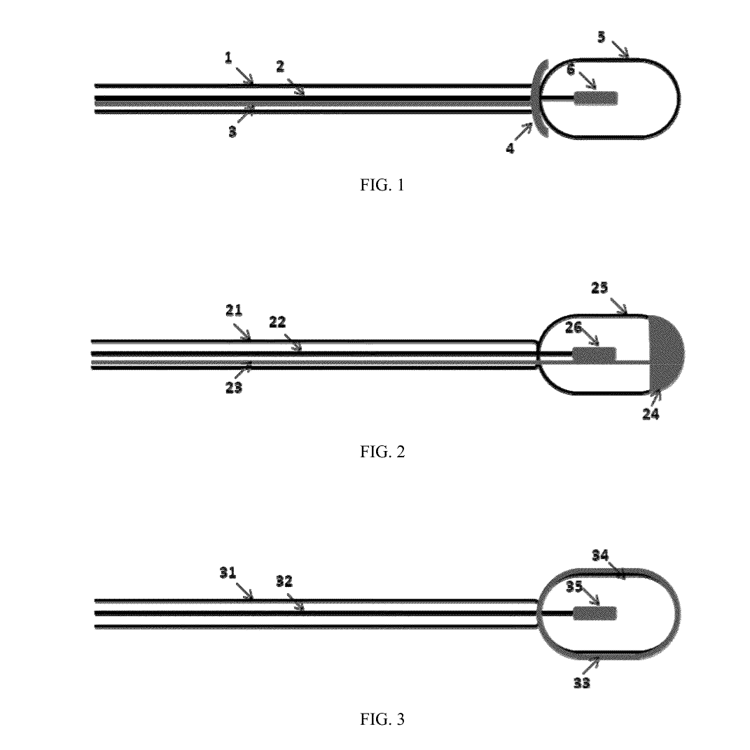

One exemplary embodiment of the present disclosure provides an apparatus to deliver the acidic liquids to the tissue under imaging, as shown in FIG. 1. In this exemplary embodiment, a hollow Tether 1 holds a Driveshaft 2 and an Acidic Liquids Delivery Duct 3. At the end of the Tether 1, an Acidic Liquids Flushing Head 4 is provided that is connected to the Acidic Liquids Delivery Duct 3, which facilitates the flushing of the acidic liquids to the tissue under imaging. The Optical Imaging Capsule 5 contains the Optical Imaging Probe 6, which can perform the microendoscopic imaging.

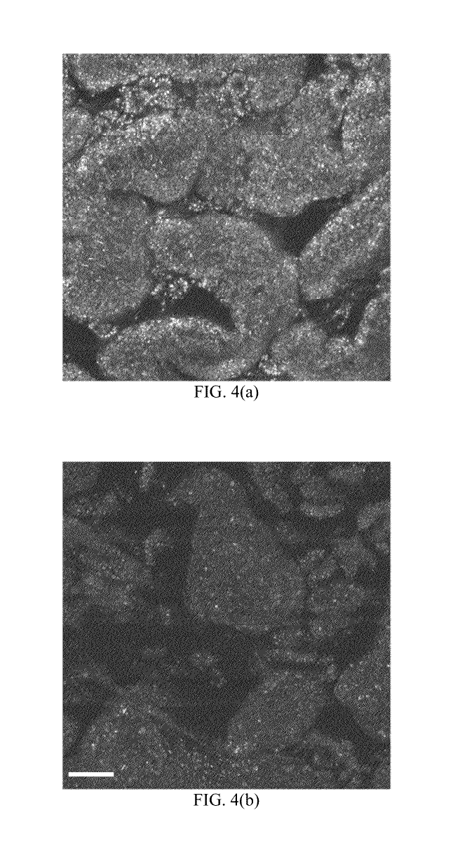

Another exemplary embodiment of the exemplary apparatus according to the present disclosure to deliver the acidic liquids to the tissue under imaging is shown in FIG. 2. For example, a hollow Tether 21 holds a Driveshaft 22 and an Acidic Liquids Delivery Duct 23. An Optical Imaging Capsule 25 can contain an Optical Imaging Probe 26, which can perform the microendoscopic imaging. At the end of the Optical Imaging Capsule 25, there is an Acidic Liquids Spray Head 24 connected to an end of the Acidic Liquids Delivery Duct 23, which facilitates a delivery of the spray of the acidic liquids to the tissue under imaging.

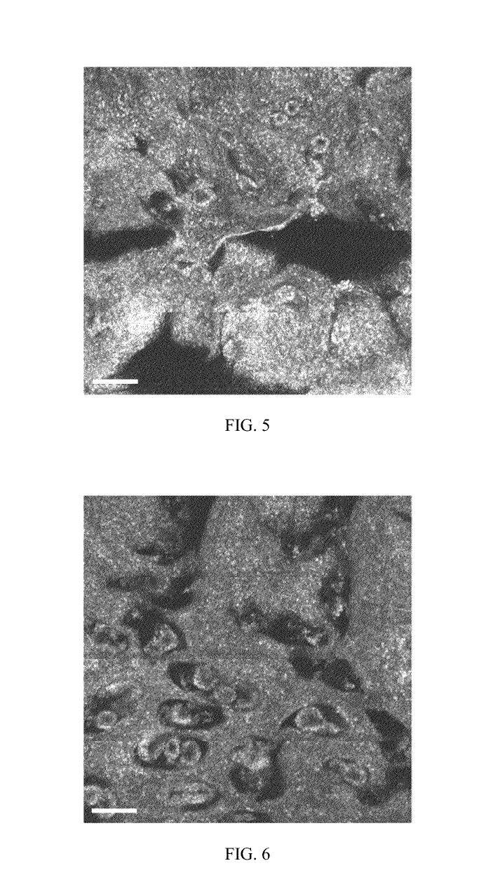

In yet further exemplary embodiment of the present disclosure, as shown in FIG. 3, acidic foods/liquids can be delivered to tissue under imaging. For example, a hollow Tether 31 can hold a Driveshaft 32. An Optical Imaging Capsule 33 can contain an Optical Imaging Probe 34, which can perform the microendoscopic imaging. Externally from the Optical Imaging Capsule 34, there can be an coating of the acidic foods/liquids with the viscous solution 34, which are made from the mixtures of the acidic foods/liquids with various concentrations of Splenda , maple syrup, corn syrup, sugar syrup, sugar, glucose, sucrose, fructose, sucralose, lactose, galactose, maltose, maltodextrin, corn starch, and guar gum, and which will be dissolved, e.g., and release the acidic foods/liquids to the tissue under imaging.

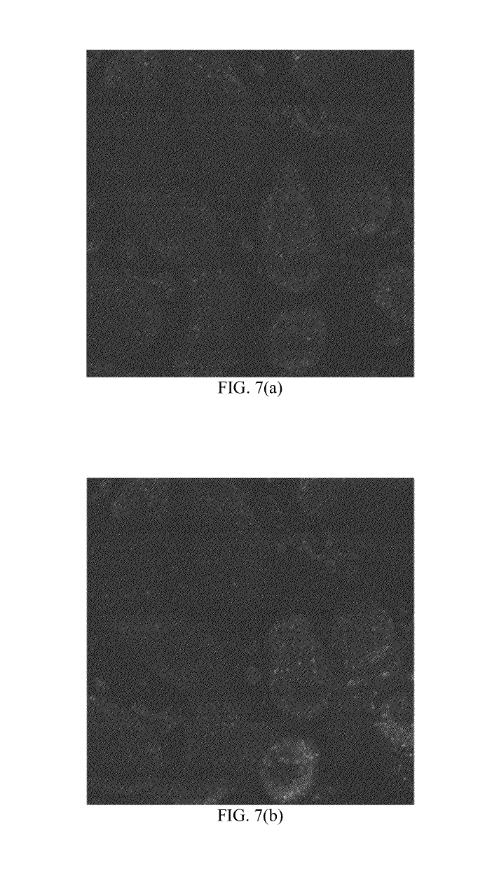

FIG. 4(a) illustrates an exemplary image taken by an exemplary SECM apparatus in swine duodenum tissue after applying Rice Vinegar, and FIG. 4(a) illustrates an exemplary image taken by the exemplary SECM apparatus in a control tissue without adding any contrast agent. The exemplary signal levels are 28.7±19.3 (a.u.) and 10.4±8.8 (a.u.) in (FIG. 4(a)) and (FIG. 4(b)), respectively. Both images of FIGS. 4(a) and 4(b) were taken at about 40 μm deep from the tissue surface. The overall confocal signal is significantly increased from all the tissue after applying Rice Vinegar. In addition, in the control image, the nuclei cannot be clearly identified. However, Rice Vinegar causes a dramatic enhancement of the nuclear contrast compared to the surrounding tissues.

It was found that, e.g., NSBR provided by Rice Vinegar is 5.0±1.1 (n=5), which is shown in Table 1, as follows:

TABLE 1
Summary of enhancement of confocal signal
by using different acidic foods/liquids

| | Agents | | | | | |
|---|---|---|---|---|---|---|
| | | | 100% | 100% | Sour | |
| | Rice | Apple | Lemon | Orange | Patch | |
| | Vinegar | Vinegar | Juice | Juice | Kids ™ | NeRds ® |
| NSBR | 5.0 ± | 4.3 ± | 4.5 ± | 2.8 ± | 3.1 ± | 3.2 ± |
| | 1.1 | 0.4 | 1.0 | 0.3 | 0.4 | 0.5 |
| | (n = 5) | (n = 5) | (n = 5) | (n = 5) | (n = 5) | (n = 5) |

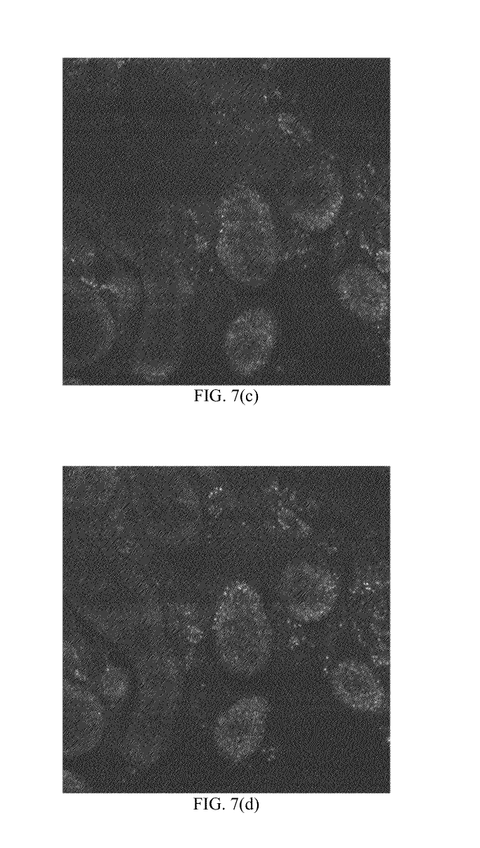

FIG. 5 shows an exemplary image taken by the exemplary SECM apparatus in swine duodenum tissue after placing Sour Patch Kids™ on its top. The exemplary image is taken at 40 μm deep from the tissue surface and the signal level is about 47.4±33.2 (a.u.). Similarly, the overall confocal signal can be significantly increased from all the tissue after placing Sour Patch Kids™ on its top. Further, the NSBR is significantly increased to about 3.1±0.4 (n=5), which is also indicated in Table 1.

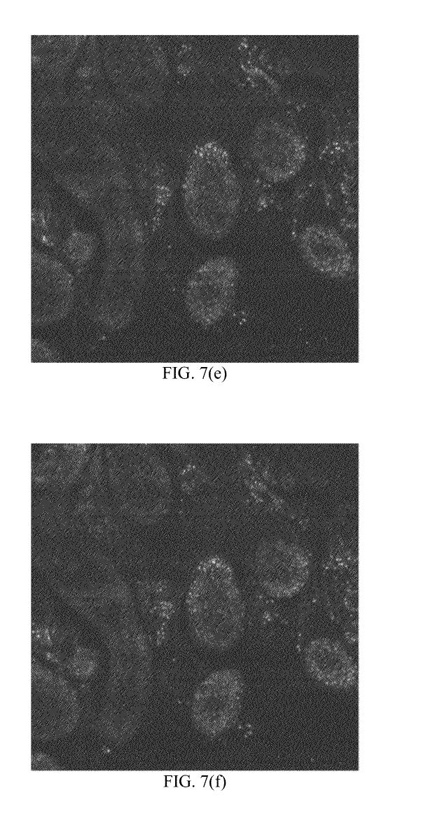

FIG. 6 shows an exemplary image taken by the exemplary SECM apparatus in swine duodenum tissue after applying mixed acidic solution of 8 g Splenda® in 4 ml Apple Vinegar. The exemplary signal level is about 26.5±16.6 (a.u.). The exemplary image is taken at about 40 μm deep from the tissue surface. Similarly, the overall confocal signal can be significantly increased from all the tissue after the application of the mixed acidic solution of 8 g Splenda in 4 ml Apple Vinegar.

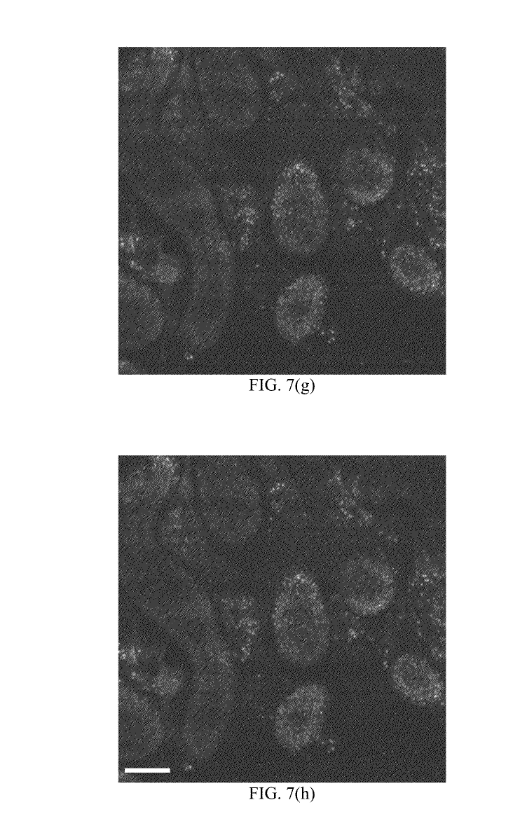

FIGS. 7(a)-7(h) show exemplary images associated with the timing of the signal enhancement induced by one of the proposed oral contrast agents. For example, FIG. 7(a) shows an image taken before applying the 100% Lemon Juice to the tissue. FIG. 7(b) shows the time when applying the applying 100% Lemon Juice to the tissue. Further, FIGS. 7(c)-(h) are the images taken at, e.g., 7 seconds, 14 seconds, 21 seconds, 28 seconds, 35 seconds, and 42 seconds, respectively, after applying the 100% Lemon Juice. Indeed, the dynamic process of the enhancement of the reflectance of the signal from the tissue has occurred based on the review of such exemplary images. Both the overall reflectance signal and the NSBR can be significantly increased.

These exemplary results indicate that, e.g., many store-bought acidic foods/liquids provide good enhancement of signal and contrast for endomicroscopic optical imaging. These exemplary acidic foods/liquids can be used as oral agents to enhance contrast for gastrointestinal tract screening in vivo, as well as contrast agents to enhance signal and contrast for respiratory tract, and other internal organs in vivo.

The foregoing merely illustrates the principles of the disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems including second or higher order harmonic microscopy, sum/difference frequency fluorescence microscopy (one-photon or multi-photon fluorescence), and Raman microscopy (CARS, SRS), and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed July 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly being incorporated herein in its entirety. All publications referenced above can be incorporated herein by reference in their entireties.

What is claimed is:

1. A system for facilitating imaging of at least one anatomical structure, comprising:
a substance which induces a change in the at least one anatomical structure when applied; and
an imaging arrangement which is configured to generate an image of the at least one anatomical structure based on the change of the at least one anatomical structure when the substance is applied.

2. The system according to claim 1, wherein the imaging arrangement further comprising an apparatus which is configured to (i) illuminate the at least one anatomical structure with a first electro-magnetic radiation(s), and (ii) receive a second electro-magnetic radiation from the at least one anatomical structure that is based on the first electro-magnetic radiation, and wherein the change effects at least one characteristic of the second electro-magnetic radiation.

3. The system according to claim 2, wherein the change is at least one of linear or nonlinear optical processes, including a reflectance, fluorescence, Raman scattering, and harmonic generation, or sum/difference frequency generation of the first electro-magnetic radiation(s) to become the second electro-magnetic radiation.

4. The system according to claim 1, wherein the imaging arrangement is a microscopic imaging arrangement.

5. The system according to claim 4, wherein the microscopic imaging arrangement operates based on at least one of confocal microscopy (SECM), optical coherence tomography ( ) second or higher order harmonic microscopy, sum/difference frequency fluorescence microscopy, or Raman microscopy (including at least one of CARS or SRS).

6. The system according to claim 5, wherein the optical coherenece tomography includes at least one of optical frequency domain interferometry (OFDI), spectral docmain optical coherence tomography (SD-OCT) or Full-field optical coherence microscopy (FFOCM).

7. The system according to claim 1, wherein the imaging arrangement has a structure that is configured to be delivered into the at least one anatomical structure.

8. The system according to claim 1, wherein the imaging arrangement has a shape of a pill and is delivered to the at least one anatomical structure.

9. The system according to claim 8, wherein a length of the pill is less than 35 mm, and a width of the pill is less than 15 mm.

10. The system according to claim 8, wherein the imaging arrangement includes a tether that is connected to the pill.

11. The system according to claim 1, wherein the imaging arrangement comprises a housing which is coated by the substance.

12. The system according to claim 1, wherein the substance includes at least one of acetic acid, tartaric acid, citric acid, malic acid, adipic acid, succinic acid, fumaric acid, hydrochloric acid, cocktail mix, or a further acidic solution which pH value is lower than 5.0.

13. The system according to claim 1, wherein the substance includes at least one of lemon juice, lime juice, orange juice, vinegar, fruit vinegar, or a further acidic juice which pH value is lower than 5.0.

14. The system according to claim 1, wherein the substance is a sour candy.

15. The system according to claim 1, wherein the substance contains miraculin.

16. The system according to claim 1, further comprising a further ingestible substance (i) which is ingested prior to the ingestion of the substance, and (ii) which alters the taste of the substance.

17. The system according to claim 1, further comprising a further ingestible substance which has a viscosity which is higher than that of water, wherein the substance and the further substance are mixed prior to ingestion.

18. The system according to claim 14, wherein the further ingestible substance includes maple syrup, corn syrup, or sugar syrup.

19. The system according to claim 14, wherein the further ingestible substance includes at least one of sugar, glucose, sucrose, fructose, sucralose, lactose, galactose, maltose, maltodextrin, corn starch, or guar gum.

20. A system for facilitating imaging of at least one anatomical structure, comprising:
a first arrangement which holds a substance therein and is configured to apply the substance to the at least one anatomical structure, wherein, upon application by the first arrangement on the at least one anatomical structure, the substance induces a change in the at least one anatomical structure; and
a second arrangement which is configured generate an image of the at least one anatomical structure based on the change of the at least one anatomical structure when the substance is applied to the at least one anatomical structure by the first arrangement, wherein at least one of the first or second arrangements are provided in a housing that has a shape of a pill, and configured to be swallowed.

21. The system according to claim 20, wherein the first and second arrangements are situated in the housing.
